(19) United States
(12) Patent Application Publication
(10) Pub. No.: US 2007/0286875 A1
(43) Pub. Date: Dec. 13, 2007

(54) ORAL LIQUID LORATADINE FORMULATIONS AND METHODS

(75) Inventors: Aparna Dagar, Deerfield, IL (US); Sivaramaprasad Gudi, San Diego, CA (US); Foyeke Opawale, Flemington, NJ (US)

Correspondence Address: WINSTON & STRAWN LLP;PATENT DEPARTMENT, 1700 K STREET, N.W., WASHINGTON, DC, 20006, US

(73) Assignee: Morton Grove Pharmaceuticals, Inc.

(21) Appl. No.: 11/759,056

(22) Filed: Jun. 6, 2007

Related U.S. Application Data
(60) Provisional application No. 60/811,418, filed on Jun. 7, 2006.

(51) Int. Cl. A61K 31/473; A61K 9/00

(52) U.S. Cl. 424/400; 514/290

(57) ABSTRACT

An oral liquid formulation including an effective amount of loratadine, or a pharmaceutically acceptable salt or metabolite thereof; and a pharmaceutically acceptable carrier including a mono- or poly-hydroxy phenol component, a solubilizing agent and a chelating agent. Methods of preparing and administering such formulations are also included.

### CROSS-REFERENCE TO RELATED APPLICATIONS

This application claims the benefit of U.S. Provisional Patent Application No. 60/811,418, filed on Jun. 7, 2006, the entire disclosure of which is hereby incorporated herein by reference.

### TECHNICAL FIELD

The present invention relates generally to formulations of oral liquid loratadine, or a pharmaceutically acceptable salt or metabolite thereof, a mono- or poly-hydroxy phenol component, and a chelating agent, and processes for their preparation and administration.

### BACKGROUND OF THE INVENTION

Loratadine is a white to off-white powder with a molecular weight of 382.89. Loratadine is not soluble in water, but is very soluble in acetone, alcohol, and chloroform.

Loratadine is a long-acting tricyclic antihistamine with selective and peripheral histamine H₁-receptor antagonistic activity. Human studies following single and repeated oral doses have shown that loratadine exhibits an antihistaminic effect beginning within 1 to 3 hours, reaching a maximum at 8 to 12 hours, and lasting in excess of 24 hours. Studies have shown no evidence of tolerance to the antihistaminic effect after 28 days of dosing. Autoradiographic, radiolabeled tissue distribution, and in vivo radioligand studies in animals have shown that neither loratadine nor its metabolites readily cross the bloodbrain barrier. Radioligand binding studies indicate preferential binding to peripheral versus central nervous system H₁-receptors.

Loratadine is also known as ethyl 4-(8-chloro-5,6-dihydro-11H-benzo[5,6]cyclohepta[1,2-b]pyridin-11-ylidene)-1-piperidinecarboxylate. Its empirical formula is C₂₂H₂₃ClN₂O₂ and it has the following structural formula:

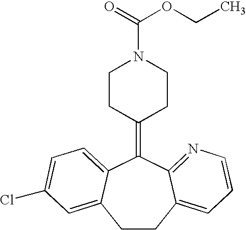

Loratadine is an active agent provided in solid and liquid oral dose forms that undergoes substantial first-pass metabolism. After oral administration, loratadine is rapidly absorbed from the gastro-intestinal tract and is metabolized to its major active metabolite, desloratadine (descarboethoxyloratadine). Based on in vitro studies with human liver microsomes, results have shown that loratadine is metabolized to descarboethoxyloratadine predominantly by cytochrome P450 3A4 (CYP3A4) and, to a lesser extent, by cytochrome P450 2D6 (CYP2D6). In the presence of a CYP3A4 inhibitor, loratadine is metabolized to descarboethoxyloratadine predominantly by CYP2D6.

Studies have shown that the pharmacokinetics of loratadine and descarboethoxyloratadine are independent of dose over the dose range of 10 mg to 40 mg and are not altered by the duration of treatment. In other studies with loratadine rapidly-disintegrating tablets, food has been shown to increase the AUC of loratadine, but did not appreciably affect the AUC of descarboethoxyloratadine. Studies have shown that the times to peak plasma concentration (Tₘₐₓ) of loratadine and descarboethoxyloratadine may be delayed when food is consumed prior to administration of loratadine rapidly-disintegrating tablets. In other pharmacokinetic studies, the AUC of loratadine was increased when administered without water compared to administration with water, while Cₘₐₓ was not substantially affected. The bioavailability of descarboethoxyloratadine was not different when administered without water.

Pharmacokinetic studies have also indicated that the mean elimination half-lives of loratadine in normal adult subjects is approximately 8.4 hours (range=3 to 20 hours), and 28 hours (range=8.8 to 92 hours) for descarboethoxyloratadine. Loratadine and descarboethoxyloratadine have been shown to reach steady-state in most patients by approximately the fifth dosing day. Approximately 40% of a dose of administered loratadine is excreted as conjugated metabolites.

Loratadine may be administered either before, with, or after administration of other therapeutic agents. Loratadine (10 mg once daily) has been co-administered with therapeutic doses of erythromycin, cimetidine, and ketoconazole in controlled clinical studies in adult volunteers. These studies demonstrated that, although increased plasma concentrations (AUC0-24 hrs) of loratadine and/or descarboethoxyloratadine were observed after coadministration of loratadine with each of these drugs, no clinically relevant changes were reported in the safety profile of loratadine. The safety profile of loratadine in these studies was assessed by different tests, including electrocardiographic parameters, clinical laboratory tests, vital signs, and incidence of adverse events. Loratadine dosage forms can also be administered in combination with other therapeutic agents, such as pseudoephedrine, such as for the treatment of cold and allergy symptoms.

Loratadine may be available for oral administration with individual doses ranging from the lowest effective dose to the recommended dose of 10 mg per day, or to a higher dose if deemed necessary by a physician, and may also be administered once or twice-daily, or according to another suitable treatment regimen. The recommended dose includes either one 10 mg tablet, or 2 teaspoonfuls (10 mg) of syrup once daily. With solid oral dosage forms, disintegration typically occurs rapidly.

A publication by R. Eyjolfsson, entitled “Loratadine: hydroxymethylation in syrup” Pharmazie (2003), 58:154, describes a loratadine syrup formulation. The Eyjolfsson publication also describes the oxidation of loratadine, and degradation products of loratadine, indicating the breakdown of loratadine in such syrup formulations. The publication also describes that the degradation of loratadine may be minimized by purging storage containers with nitrogen or including edetate disodium in the formulation.

U.S. Pat. No. 6,132,758 describes stabilized syrup formulations that include loratadine formulations. The '758 patent describes active agent stability in aqueous media, and reports that trace metal-initiated oxidation reactions can be minimized through the potential use of citric acid or certain sequestering agents. The '758 patent also describes other antioxidant additives for incorporation into syrup formulations. Ascorbic acid was described to reduce degradation, but caused an unacceptable strong color change in the product, while sodium bisulfite was described as imparting a pungent odor to a syrup formulation.

Loratadine is available by prescription in tablet or syrup form as a sole active ingredient (Claritin®, Schering). Claritin® is indicated for the relief of nasal and non-nasal symptoms of seasonal allergic rhinitis and for the treatment of chronic idiopathic urticaria. Claritin® is available for oral administration as tablets, including rapidly-disintegrating tablets, containing 10 mg loratadine. Claritin® tablets contain 10 mg micronized loratadine, and the following inactive ingredients: corn starch, lactose, and magnesium stearate. Claritin® RediTabs® (loratadine rapidly-disintegrating tablets) contain 10 mg micronized loratadine, and also contain the following inactive ingredients: citric acid, gelatin, mannitol, and mint flavor. Claritin® is also available as a clear, colorless to light-yellow syrup, containing 1 mg loratadine per mL. Claritin® Syrup contains micronized loratadine (1 mg/mL), and the following inactive ingredients: citric acid, edetate disodium, artificial flavor, glycerin, propylene glycol, sodium benzoate, sugar, and water.

Because of the ease of preparing solid dosage forms, tablets and capsules are often the preferred dosage form for many drugs. Liquid dosage forms present more of a challenge because of the solubility and stability characteristics of the active compound, as well as the various excipients, in liquid form. In particular, liquid dosage forms present a challenge because of difficulties with stability due to oxidative degradation of the active compound, as well as bitterness or other taste problems that tend to reduce patient compliance. In view of the foregoing, it would be desirable to have increase the stability and/or dissolution of oral liquid loratadine compositions.

### SUMMARY OF THE INVENTION

The invention encompasses an oral liquid formulation including a therapeutically or prophylactically effective amount of loratadine, or a pharmaceutically acceptable salt or metabolite thereof, and a pharmaceutically acceptable carrier that includes a poly- or mono-hydroxy phenol component in an amount sufficient to increase stability of the loratadine, a solubilizing agent present in an amount sufficient to facilitate dissolution of the loratadine and the phenol component, and a chelating agent including at least one organic acid in an amount sufficient to increase dissolution of the phenol component and loratadine and to increase stability of the loratadine.

In a preferred embodiment, the phenol component includes butylated hydroxyanisole. In another embodiment, the sufficient amount of phenol component is from about 0.05 mg/5 mL to 5 mg/5 mL of the formulation. In another embodiment, the organic acid includes one or more of acetic acid, propionic acid, butyric acid, a fatty acid of 6-22 carbon atoms, bile acid, lactic acid, citric acid, pyruvic acid, oxalic acid, malic acid, malonic acid, succinic acid, maleic acid, fumaric acid, tartaric acid, benzoic acid, cinnamic acid, mandelic acid and salicylic acid. In yet another embodiment, the sufficient amount of the chelating agent is from about 1 mg/5 mL to 150 mg/5 mL of the formulation.

In a preferred embodiment, the solubilizing agent includes a glycol. In a preferred embodiment, this is present in an amount of about 4 to 15 volume percent. In a preferred embodiment, the glycol includes propylene glycol. In another embodiment, the pharmaceutically acceptable carrier further includes one or more of a stabilizing agent, thickening agent, sweetening agent, flavoring agent, colorant agent, preservative agent, antioxidant agent, or buffering agent. In a preferred embodiment, the carrier further includes at least one of a sweetening agent, a flavoring agent, and a preservative agent. Preferably, it includes at least two, and more preferably all three, of the sweetening, flavoring, and preservative agents. In one embodiment, the sweetening agent includes glycerin, sucrose, liquid sugar, sorbitol, saccharin, xylitol, maltitol, an acesulfame-containing, sucralose-containing or saccharin-containing component, or a combination thereof; and the flavoring agent comprises grapefruit, orange, lemon, lime, mango, strawberry, banana, pineapple, cherry, or a combination thereof. In a preferred embodiment, the sweetening agent is present in an amount of about 1 volume percent to 85 volume percent (v/v).

In another preferred embodiment, the sweetening agent includes liquid sugar and glyercin. In another preferred embodiment, the preservative agent includes one or more of sodium benzoate, chlorobutanol, benzyl alcohol, and benzalkonium chloride. In another preferred embodiment, the preservative agent is present in an amount of about 0.05 mg/5 mL to 10 mg/5 mL. In yet another preferred embodiment, the flavoring agent is present in an amount of about 0.01 volume percent to 1 volume percent (v/v). In a more preferred embodiment, all of the sweetening, flavoring, and preservative agents are present and the sweetening agent comprises glycerin and liquid sugar in an amount of about 20 volume percent to 70 volume percent (v/v); the flavoring agent comprises strawberry, banana, pineapple, or a combination thereof, in an amount of about 0.01 volume percent to 1 volume percent (v/v); and the preservative agent comprises sodium benzoate in an amount of about 0.1 mg/5 mL to 10 mg/5 mL.

In yet another embodiment, the therapeutically or prophylactically effective amount of loratadine, or salt or metabolite thereof, is a concentration of about 0.1 mg/5 mL to 20 mg/5 mL of the formulation. In one preferred embodiment, the formulation is at least substantially stable. In yet another preferred embodiment, the degradation of loratadine over a period of up to three months is no more than about 1 percent to 2 percent (w/v) at 40° C.

The invention also encompasses a stable oral liquid formulation including a therapeutically or prophylactically effective amount of loratadine, or a pharmaceutically acceptable salt or metabolite thereof, and a pharmaceutically acceptable carrier including butylated hydroxyanisole, and a chelating agent that includes at least one of citric acid anhydrous, acetic acid, propionic acid, butyric acid, a fatty acid of 6-22 carbon atoms, bile acid, lactic acid, pyruvic acid, oxalic acid, malic acid, malonic acid, succinic acid, maleic acid, fumaric acid, tartaric acid, benzoic acid, cinnamic acid, mandelic acid and salicylic acid, wherein the butylated hydroxyanisole and the chelating agent are each present in amount sufficient to synergistically increase the stability of the loratadine. In a preferred embodiment, the effective amount of loratadine is from about 1 mg/5 mL to 20 mg/5 mL of loratadine, or a pharmaceutically acceptable salt or metabolite thereof, the chelating agent includes citric acid; and the formulation further includes a preservative agent, a sweetening agent, and a flavoring agent.

In another embodiment, the invention encompasses a stable oral liquid formulation including a therapeutically or prophylactically effective amount of loratadine, or a pharmaceutically acceptable salt or metabolite thereof, and a pharmaceutically acceptable carrier including a mono- or poly-hydroxy phenol component in an amount sufficient to increase stability of the loratadine, a chelating agent comprising at least one organic acid in an amount sufficient to increase dissolution of the phenol component and loratadine and to increase stability of the loratadine, and propylene glycol in an amount sufficient to increase dissolution of the phenol component and loratadine.

The invention also encompasses a method of preparing a stable oral liquid loratadine formulation by providing a pharmaceutically acceptable carrier including a mono- or poly-hydroxy phenol component in an amount sufficient to increase stability of the formulation, and a chelating agent including at least one organic acid in an amount sufficient to increase dissolution of the phenol component and to increase stability of the formulation, and dissolving a therapeutically or prophylactically effective amount of loratadine, or a pharmaceutically acceptable salt or metabolite thereof, into a portion of the pharmaceutically acceptable carrier so as to provide the stable oral liquid loratadine formulation.

In one preferred embodiment, the phenol component includes butylated hydroxyanisole. In a more preferred embodiment, the carrier includes butylated hydroxyanisole and citric acid anhydrous. In another preferred embodiment, the oral liquid loratadine formulation can be clear or translucent.

The invention further encompasses methods preventing, treating, or managing allergic symptoms in a mammal by administering to the mammal a therapeutically or prophylactically effective amount an oral liquid loratadine formulation prepared according to the invention. In one embodiment, the formulation is administered once or twice a day as a syrup.

In another embodiment, the total daily dose of loratadine is from about 5 mg to 50 mg. In yet another embodiment, the method further includes administering an effective amount of at least one other therapeutic agent.

The invention also encompasses a liquid formulation container including the oral liquid loratadine formulation of the invention disposed in a substantially non-air permeable bottle, wherein the formulation fills greater than about 90% of the bottle so as to reduce container headspace and to decrease oxidative degradation of the loratadine. In a preferred embodiment, the substantially non-permeable container, such as a glass container, is used to reduce degradation of loratadine.

It should be understood that each of the embodiments described herein is applicable to each aspect of the invention.

### DETAILED DESCRIPTION OF THE PREFERRED EMBODIMENTS

The present invention surprisingly provides a therapeutically effective or prophylactically effective amount of loratadine, or a pharmaceutically acceptable salt or metabolite thereof, in an oral liquid formulation that is preferably substantially or entirely stable.

This is achieved by including a pharmaceutically acceptable carrier that increases the dissolution and stability of loratadine and thus reduces the degradation of loratadine. Preferably, the carrier includes at least one mono- or poly-hydroxy phenol component in an amount sufficient to increase stability of the loratadine, at least one solubilizing agent present in an amount sufficient to facilitate dissolution of the loratadine and the phenol component, and a chelating agent including at least one organic acid in an amount sufficient to increase dissolution of the phenol component and loratadine and to increase stability of the loratadine.

As used herein, “oral liquid solution(s)” is a preferred embodiment of the liquid formulations and the term “solution” is used merely as an exemplary type of formulation. The term “formulation” or any other type of formulation is substitutable for the term “solution” as used herein, although solutions and syrup solutions are a preferred type of formulation. “Oral liquid solution(s)” do not include solid dosage forms that include minor amounts of solutions or other liquid components therein, such as filled capsules. Preferably, the oral liquid solutions, i.e., formulations, are substantially free of undissolved loratadine, carrier, or both, more preferably both. Preferably, an at least substantially stable oral liquid loratadine solution may be prepared that is substantially free of impurities, and more preferably entirely free of impurities.

The present liquid dosage forms provide certain advantages over the solid forms conventionally available. For example, liquid dosage forms are much easier to swallow, and tend to increase patient compliance. The present liquid dosage forms may also be prepared as syrups for administration. Patient compliance is typically further increased by providing non-bitter flavoring or interesting colorant agents that are not typically included in solid dosage forms. The suitability of dosage forms and patient compliance are often an issue with very young patients and the elderly. For any patient likely to suffer from allergies, symptoms of seasonal allergic rhinitis, or other symptoms or conditions associated with histamine release and the effects of histamine, for which loratadine may be prescribed, it would be beneficial to have a palatable oral solution that is at least substantially stable and can help increase patient compliance.

The active ingredient in the present invention is loratadine. Loratadine is preferably used alone, i.e., not in the form of a salt. Alternatively, it can be used in the form of a pharmaceutically acceptable salt or metabolite that retains the biological effectiveness and properties of loratadine and is not biologically or otherwise undesirable. As used herein, “loratadine” includes the agent itself, or an active metabolite or pharmaceutically acceptable salt of either, or any combination thereof. “Loratadine” also includes polymorphs of the active ingredient. For example, a polymorph of ethyl 4(8-chloro-5,6-dihydro-11H-benzo[5,6]cyclohepta[1,2-b]pyridin-11-ylidene)-1-piperidenecarboxylate (loratadine), which has been described in U.S. Pat. No. 6,335,347, and which is incorporated herein by express reference thereto, can be included as the claimed loratadine. “Salt” used in connection with loratadine herein refers to a pharmaceutically acceptable salt.

Loratadine may form acid addition salts and salts with bases. Although any available salt made by any method available to those of ordinary skill in the art may be used, a few exemplary acids and bases are described. Exemplary acids that can be used to form such salts include mineral acids such as hydrochloric, hydrobromic, sulfuric or phosphoric acid; or organic acids such as organic sulfonic acids and organic carboxylic acids; and any combination thereof. Salts formed with inorganic bases include, for example, the sodium, potassium, lithium, ammonium, calcium, and magnesium salts, or any combination thereof. Salts derived from organic bases include, for example, salts of primary, secondary and tertiary amines, substituted amines including naturally-occurring substituted amines, and cyclic amines, including isopropylamine, trimethylamine, diethylamine, triethylamine, tripropylamine, ethanolamine, 2-dimethyl aminoethanol, trimethamine, lysine, arginine, histidine, caffeine, procaine, hydrabamine, choline, betaine, ethylenediamine, glucosamine, N-alkylglucamines, theobromine, purines, piperazine, piperidine, N-ethylpiperidine, fumarate, maleate, succinate, acetate oxalate, or a combination thereof. A preferred loratadine salt includes a hydrochloric salt.

Suitable pharmaceutically acceptable salts of loratadine may also be prepared. For example, the preparation of a suitable salt of loratadine can be achieved using the methods of, for example, U.S. Pat. No. 6,110,927, which is incorporated herein by express reference thereto.

Suitable metabolites of loratadine may also be prepared. For example, U.S. Pat. No. 6,110,927 also describes methods for the preparation of descarboethoxyloratadine. In another example, the preparation of desloratadine is described in U.S. Patent Application Publication No. 2005/0203116, which is incorporated herein by express reference thereto.

In one preferred embodiment, the loratadine agent together with the carrier, forms a solution. Preferably, the loratadine is micronized to facilitate dissolution in the liquid formulation of the invention. Typically, substantially all the loratadine and carrier are in solution, and preferably all the loratadine and carrier are in solution. Indeed, it is preferred that a solution of the present invention be at least substantially free of undissolved particulates or other impurities. Preferably, the solution is entirely free of any undissolved particulates or other impurities. Therefore, the carrier preferably acts as a solvent for loratadine. In another preferred embodiment, the loratadine, or salt or metabolite thereof, together with the carrier, forms a syrup.

Preferably, an effective amount of loratadine in an oral liquid solution is a concentration of about 0.1 mg/5 mL to 20 mg/5 mL of the solution. In more preferred embodiment, the effective amount of loratadine includes amounts from about 1 mg/5 mL to 15 mg/5 mL of the solution or from about 3 mg/5 mL to 12 mg/5 mL. An exemplary amount of loratadine according to the invention is from about 4 mg/5 mL to 10 mg/5 mL of the solution.

Preferably, the carrier includes one or more compounds for increasing the stability of the loratadine in solution. Preferably, however, the carrier includes any suitable monohydroxy phenol component or polyhydroxy phenol component, or a combination thereof available to those of ordinary skill in the art. In one embodiment, a monohydroxy phenol component is selected, and may preferably include one or more methoxyphenol compounds. It is to be understood that the monohydroxy phenol component and/or polyhydroxy phenol component according to the invention may also function as a stabilizing agent, an antioxidant agent, an antimicrobial agent, or any combination thereof, and can be included in or as the stabilizing or antioxidant agent or antimicrobial agent, or any combination thereof.

A particularly preferred example of a monohydroxy phenol component includes butylated hydroxyanisole (BHA), i.e., tert-butyl-4-methoxyphenol. BHA has anti-oxidant properties and may reduce, and preferably substantially reduce, oxidative degradation of loratadine. BHA may function as an antioxidant by minimizing, or substantially minimizing, free-radical-mediated chain reactions. BHA has the following structure:

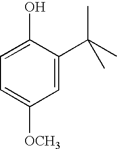

The carrier may also include any suitable polyhydroxy phenol component in an amount sufficient to increase stability of the loratadine in solution. In one preferred example, the carrier may include propyl gallate, a polyhydroxy phenol component, which may also function as an antioxidant agent. It is to be understood that any suitable monohydroxy or polyhydroxy phenol component may also have any number of various ring substitutions. Preferably, the monohydroxy or polyhydroxy phenol component is not soluble only in oil, and may be dissolved at least to some extent, and preferably completely dissolved, in an aqueous medium. Preferred phenol components dissolve at least substantially in aqueous media. Thus, in one embodiment, the carrier preferably will not include a mono- or poly-hyroxy phenol component that includes a substantial amount of any poorly soluble or insoluble compounds, such as butylated hydroxytoluene (BHT), i.e., 3,5-di-tert-butyl-4-hydroxytoluene or tertiary butylatedhydroquinone (TBHQ), i.e., tert-butyl-1,4-benzenediol.

According to one embodiment, the carrier typically includes a monohydroxy phenol compound in an amount from about 0.05 mg/5 mL to 5 mg/5 mL of the solution. Preferably, the carrier includes a monohydroxy phenol compound in an amount from about 0.1 mg/5 mL to 2 mg/5 mL, based on the total volume of the solution. More preferably, the monohydroxy phenol compound may be present in an amount from about 0.25 mg/5 mL to 0.75 mg/5 mL, based on the total volume of the solution. Preferably, the monohydroxy phenol component and the chelating agent are each present in an amount sufficient to produce a synergistic increase in stability of the loratadine.

Preferably, the carrier also includes a chelating agent to increase the stability of the loratadine in solution. A chelating agent may be added to trap metals that find their way into the compositions, such as during processing, and thereby reduce metal-mediated oxidation of loratadine. The chelating agent preferably includes one or more organic acids. Examples of suitable organic acids are acetic acid, propionic acid, butyric acid, a fatty acid of 6-22 carbon atoms, bile acid, lactic acid, citric acid, pyruvic acid, oxalic acid, malic acid, malonic acid, succinic acid, maleic acid, fumaric acid, tartaric acid, benzoic acid, cinnamic acid, mandelic acid and salicylic acid, or a combination thereof. A preferred chelating agent includes citric acid. In one preferred embodiment, the organic acid(s) are anhydrous. Examples of other suitable materials that can be included in the chelating agent include, but are not limited to, ethylenediamine (EDA), diethylenetriamine (DETA), aminoethylethanolamine (AEEA), and combinations thereof. Preferably, the chelating agent is at least substantially free, and more preferably entirely free, of EDTA. Any suitable chelating agent can be included so long as it does not prevent the dissolution or stability of the loratadine. It is to be understood that chelating agents according to the invention may also function as a stabilizing agent, an antioxidant agent, or both, and can be included in or as the stabilizing or antioxidant agent, or both.

The carrier may preferably include a chelating agent in an amount sufficient to increase the stability of the loratadine in solution. Preferably, the carrier may include an amount of a chelating agent that is sufficient to facilitate the solubility of one or more monohydroxy phenol compounds in the solution, and also to increase the stability of the loratadine in the solution. As a mechanism to reduce degradation, it is believed that the chelating agent may reduce metal-mediated oxidation of loratadine. Preferably, a chelating agent may be present in an amount from about 1 mg/5 mL to 150 mg/5 mL of the solution. Preferably, the chelating agent may be present in an amount from about 10 mg/5 mL to 120 mg/5 mL of the solution. Preferably, the carrier may include a chelating agent in an amount from about 40 mg/5 mL to 100 mg/5 mL, and more preferably, from about 50 mg/5 mL to 90 mg/5 mL, based on the total volume of the solution.

According to one preferred embodiment, the carrier includes BHA and an organic acid-based chelating agent. More preferably, loratadine may be solubilized in a carrier including BHA and citric acid anhydrous. Preferably, the carrier or even the entire solution are at least substantially, or more preferably entirely, free of oil under ambient conditions. Thus, in a preferred aspect, the invention encompasses aqueous loratadine solutions.

An exemplary oral liquid solution might include 5 mg/5 mL of loratadine (including possibly a salt or metabolite thereof, as with all loratadine described herein) in a solution that includes from about 50 mg/5 mL to 100 mg/5 mL of citric acid anhydrous, and about 0.3 mg/5 mL to 0.5 mg/5 mL of BHA. Preferably, the oral liquid loratadine formulation is at least substantially stable. In another preferred embodiment, the BHA may have synergistic effects with one or more chelating agents, e.g., citric acid, to further increase the stability of the loratadine. In one embodiment, the degradation of loratadine over a period of less than three months is no more than about 1 percent to 2 percent (w/v) at 40° C.

The pharmaceutically acceptable carrier typically can also include one or more optional additional additives to advantageously modify one or more formulation properties, including a pH modifying agent (e.g., buffering agent), stabilizing agent, thickening agent, sweetening agent, flavoring agent, colorant agent, preservative agent, emulsifying agent, solubilizing agent, antioxidant agent, or any combination thereof. Any reference to “carrier” herein should be understood to refer to pharmaceutically acceptable carriers. In the case of each type of carrier, any suitable selection or amount available to those of ordinary skill in the art may be included according to the invention so long as the carrier as a whole does not significantly detrimentally affect the stability of the loratadine. Preferably, the carrier includes one or more of a solubilizing agent, a stabilizing agent, a sweetening agent, a flavoring agent, and a preservative agent, in addition to the monohydroxy phenol component, chelating agent, and loratadine.

Preferably, the carrier is suitable for forming an aqueous oral liquid loratadine composition. The carrier is selected to facilitate dissolution of substantially all the loratadine, whether or not added water is present. Alternatively, in one preferred embodiment, the oral loratadine solution is at least essentially, or entirely, aqueous (i.e., added water is present) at the time the patient begins consuming the medication over a course of treatment.

A thickening agent or viscosity-enhancing agent may be included in the carrier to generally improve the mouth-feel of the composition and/or to help coat the lining of the gastrointestinal tract. While any suitable thickening agent available to those of ordinary skill in the art may be included in the compositions of the present invention, a preferred thickening agent, when used, includes acacia, alginic acid bentonite, carbomer, carboxymethylcellulose calcium or sodium, cetostearyl alcohol, methyl cellulose, ethylcellulose, glycerin, gelatin guar gum, hydroxyethyl cellulose, hydroxymethyl cellulose, hydroxypropyl cellulose, hydroxypropyl methyl cellulose (“HPMC”), any other suitable cellulose-based component, maltodextrin, polyvinyl alcohol, povidone, propylene carbonate, propylene glycol alginate, sodium alginate, sodium starch glycolate, starch tragacanth, and xanthan gum, or a combination thereof. A preferred type of thickening agent includes a cellulose-based component, which includes many of the cellulosic materials noted above.

Typical amounts of thickening agent, when included, are present in an amount of about 0.1 volume percent to 20 volume percent (v/v), based on the total volume of the solution. In one example, glycerin is present in an amount of about 1 volume percent to 10 volume percent (v/v), based on the total volume of the solution. Exemplary amounts of thickening agent include from about 1 volume percent to 12 volume percent (v/v), and preferably at an amount of about 4 volume percent to 10 volume percent (v/v), based on the total volume of the solution. An exemplary amount includes about 6 to 10 volume percent (v/v).

A sweetening agent is optionally but preferably included in the carrier. Any suitable sweetening agent available to those of ordinary skill in the art may be used according to the invention. Typically, when present the sweetening agent includes sorbitol, saccharin, acesulfame, e.g., acesulfame potassium, sucralose, xylitol, maltitol, sucrose, aspartame, fructose, neotame, glycerin, sodium saccharate, glycyrrhizin dipotassium, acesulfame K, mannitol, invert sugar, and combinations thereof, or components containing a sweetening agent, such as one or more sucralose-containing components or saccharin-containing components, may be added to modify the taste of the composition. Alternatively, or in addition, a viscous sweetener such as one or more of a sorbitol solution, a syrup (sucrose solution), or high-fructose corn syrup can be used and, in addition to sweetening effects, may also be useful to increase viscosity and to retard sedimentation. In one embodiment, the sweetening agent including an acesulfame-containing, sucralose-containing, or saccharin-containing component. Preferably, the sweetening agent includes glycerin, saccharin, liquid sugar (sucrose solution), or a combination thereof. Such a sweetening agent, if present, may be present in an amount sufficient to minimize or mask any off-flavors in the taste of the loratadine, or salt or metabolite thereof, and preferably also to minimize or mask any other off-flavor components included in the formulation if desired.

Typical amounts of sweetening agent, when included, are present in an amount of about 0.1 volume percent to 85 volume percent (v/v), based on the total volume of the solution. In one example, the sweetening agent is present in an amount of about 5 volume percent to 70 volume percent (v/v), based on the total volume of the solution. Exemplary amounts of glyercin include about 2 volume percent to 18 volume percent (v/v), preferably about 5 volume percent to 10 volume percent (v/v). Exemplary amounts of liquid sugar may include about 40 volume percent to 75 volume percent (v/v), preferably about 60 volume percent to 70 volume percent (v/v), based on the total volume of the solution.

Certain types of thickening agent or sweetening agent may also act as a solubilizing agent or a stabilizing agent, or both, or have other properties, when included as a component of a pharmaceutically acceptable carrier. For example, a sweetening agent such as glycerin may also act as a thickening agent. An oral liquid loratadine composition may also contain, in addition to a sweetening agent, a flavoring agent, for example, one or more of natural and artificial fruit, artificial banana, strawberry, and pineapple.

Any suitable flavoring agent available to those of ordinary skill in the art may be included in the present loratadine solutions, typically to enhance patient compliance by making the compositions of the present invention more palatable. The flavoring agent is typically selected in type and amount to increase palatability, e.g., by decreasing or eliminating any undesired taste or off-flavors in the taste, i.e., a taste mask, that would otherwise be detectable by a typical patient to whom the compositions are administered. Examples of a suitable flavoring agent, when used, include one or more of menthol, peppermint, anise, and any fruit flavor, such as one or more of grapefruit, orange, banana, lemon, lime, mango, strawberry, pineapple, or cherry, natural and artificial fruit mix flavor, or a combination thereof.

Typical amounts of flavoring agent, which is optional but preferred, may be present in the carrier in an amount of about 0.05 volume percent to 1.5 volume percent (v/v), based on the total volume of the solution. In one example, the flavoring agent, e.g., artificial banana flavor, is present in an amount of about 0.1 volume percent to 1 volume percent (v/v), based on the total volume of the solution. Exemplary amounts of flavoring agent include about 0.2 volume percent to 0.8 volume percent (v/v) or an amount of about 0.4 volume percent to 0.6 volume percent (v/v), based on the total volume of the solution.

A colorant agent, when included in the carrier, may be provided in an amount sufficient to provide the compositions with a more aesthetic and/or distinctive appearance. Any suitable colorant agent available to those of ordinary skill in the art may be selected. Typically, a colorant agent suitable for inclusion in the present invention includes one or more synthetic organic food additives (e.g., food dyes such as food red dye Nos. 2 and 3, food yellow dye Nos. 4 and 5 and food blue dye Nos. 1 and 2), water-insoluble lake dyes (e.g., aluminum salts of the above synthetic organic food additives, etc.), and natural pigments (e.g., beta-carotene, chlorophyll, iron oxide red, etc.). Other suitable colorants include D&C Red No. 33, FD&C Red No. 3, FD&C Red No. 40, D&C Yellow No. 10, and C Yellow No. 6, or any combination of these or the above colorants.

It is optional, but preferred, to include a suitable preservative agent in the carrier. When included, any preservative agent available to those of ordinary skill in the art may be included, typically in an amount sufficient to extend the shelf-life or storage stability, or both, of the present loratadine solutions. Preferred examples of a suitable preservative agent, when used, include sodium benzoate, paraoxybenzoic acid esters, methyl, ethyl, butyl, and propyl parabens, chlorobutanol, benzyl alcohol, phenylethylalcohol, dehydroacetic acid, sorbic acid, benzalkonium chloride (BKC), benzethonium chloride, phenol, phenylmercuric nitrate, thimerosal, and a combination thereof. A more preferable preservative agent includes sodium benzoate.

A preservative agent may be added to the carrier at levels safe for ingestion. Typical amounts of preservative agent, when included, may be from about 0.05 mg/5 mL to 10 mg/5 mL, based on the total volume of the solution. In one example, the preservative agent is present in an amount of about 0.2 mg/5 mL to 8 mg/5 mL, based on the total volume of the solution. Exemplary amounts of preservative agent include about 0.3 mg/5 mL to 5 mg/5 mL, based on the total volume of the solution.

The loratadine solutions typically have a pH of about 2.5 to 3.1. The pH of the oral liquid composition may be adjusted by a buffering agent. The buffering agent, when used, is typically present in an amount sufficient to buffer the pH of the solution and minimize degradation of the loratadine. It may also modulate drug solubility in the inventive solutions. While any suitable buffering agent available to those of ordinary skill in the art can be included in the carrier, the buffering agent preferably includes one or more of gluconate, lactate, citrate, acetate, phosphate, benzoate, and/or carbonate salts. Preferably, the pH can be adjusted with a combination of two or more of these buffering agents, e.g., citric acid and sodium benzoate. The buffering agent can be present as a buffer solution, particularly for oral liquid formulations that include a solution. In another example, the buffering agent may include a phosphate, more preferably a potassium phosphate or sodium phosphate, or a combination thereof.

Emulsifying agents can be used in the carrier an amount sufficient to facilitate more uniform dispersion of an active ingredient or other excipient that is not generally soluble in the liquid carrier. Although any suitable emulsifying agent available to those of ordinary skill in the art can be used, if present a preferred emulsifying agent includes gelatin, egg yolk, casein, cholesterol, acacia, tragacanth, chondrus, pectin, methyl cellulose, carbomer, cetostearyl alcohol, cetyl alcohol, or a combination thereof.

Solubilizing agents can optionally but preferably be included, for example, in the carrier in an amount sufficient to facilitate greater or more rapid dissolution of an active ingredient or other excipient. Preferably, when included, the solubilizing agent is present in an amount sufficient to facilitate dissolving or dispersing the loratadine, or salt or metabolite thereof, in the carrier. While any suitable solubilizing agent available to those of ordinary skill in the art can be included in the present formulations, preferably the solubilizing agent may include an alcohol, e.g., 95% ethyl alcohol, a glycol, glycerin, D-mannitol, trehalose, benzyl benzoate, trisaminomethane, cholesterol, triethanolamine, sodium carbonate, sodium citrate, sodium salicylate, sodium acetate, and a combination thereof. Preferred alcohols include ethanol, isopropanol, t-butanol, phenol, cresol, a benzyl alcohol, or a combination thereof. Preferably, the solubilizing agent may include a glycol. Suitable glycols may include, for example, those C₂₋₂₀ alkenes functionalized with a glycol, including propylene glycol, polypropylene glycol, polyethylene glycol, etc., or a combination thereof. Preferred glycols include polyethylene glycol, such as PEG-400, or propylene glycol, or both. More preferably, an aqueous oral liquid loratadine composition includes the desired amount of loratadine component, for example, 5 mg/5 mL of loratadine, along with a carrier that includes from about 45 mg/5 mL to 100 mg/5 mL of citric acid anhydrous, about 0.3 mg/5 mL to 0.5 mg/5 mL of BHA, and about 4 volume percent to about 15 volume percent propylene glycol (v/v), based on the total volume of the solution.

Typical amounts of solubilizing agent, when included, are present in an amount of about 1 volume percent to 20 volume percent (v/v), and more preferably about 4 volume percent to 15 volume percent (v/v), based on the total volume of the solution. Exemplary amounts of solubilizing agent include about 7 volume percent to 12 volume percent (v/v) based on the total volume of the solution.

A stabilizing agent can include any suitable agent that increases the stability of loratadine. The stabilizing agent can include, for example, one or more liquid excipients such as ethanol, glycerin; one or more glycols, such as polyethylene glycol, e.g., PEG-400, propylene glycol, or polypropylene glycol; a cellulose-based component, such as hydroxypropylmethylcellulose (HPMC) or hydroxymethylcellulose (HMC); or any combination thereof. Thus, it should be understood that certain solubilizing agents may function effectively as a stabilizing agent. For example, propylene glycol may function as both a solubilizing agent and as a stabilizing agent.

Examples of a suitable antioxidant, if used, include one or more flavonoids, anthocyanidins, anthocyanins, proanthocyanidins, and combinations thereof. Preferably, the formulation is at least substantially free, and more preferably entirely free, of sodium bisulfite or metabisulfite, which may (without being bound by theory or preliminary testing) cause undesirable odors, flavors, or other disadvantageous side effects. The antioxidant, when used, can help provide long term stability to the liquid compositions, e.g., at ambient conditions for at least about one month, preferably for at least about 3 months, and more preferably for at least about 24 months, or longer, depending on the type and concentration of antioxidant used and depending on other components of the storage microenvironment, such as pH, buffering agent, etc. Even at elevated temperatures, e.g., at least 40° C., the liquid compositions of the invention are stable for at least about three months, preferably at least about 6 months. In one embodiment, a suitable amount of the antioxidant component, if present, is about 0.01 mg/5 mL to 1 mg/5 mL, preferably about 0.1 mg/5 mL to 0.8 mg/5 mL, and more preferably about 0.2 mg/5 mL to 0.6 mg/5 mL, based on the total volume of the solution.

Through selection and combination of the pharmaceutically acceptable carrier according to the invention, liquid loratadine compositions may be provided that exhibit improved or more desired performance with respect to drug concentration, dissolution, dispersion, stability, safety, emulsification, efficacy, flavor, patient compliance, bioavailability, and/or other pharmacokinetic, chemical and/or physical properties. In one preferred embodiment, a therapeutically or prophylactically effective amount of loratadine, or a pharmaceutically acceptable salt or metabolite thereof, can be advantageously dissolved to generate a substantially stable, or stable, solution with a pharmaceutically acceptable carrier as described herein.

A few exemplary embodiments of the invention are now set forth. In a first exemplary embodiment, the composition includes about 5 mg/5 mL loratadine, or a salt or metabolite thereof, in a solution that includes about 65 mg/5 mL of citric acid anhydrous and about 0.375 mg/5 mL of BHA. As used herein, the terms (v/v) and (w/v) refer to percentages based on volume and percentages based on weight per volume, respectively. Furthermore, it is to be understood that amounts of agents or components are based on either a mg/mL (w/v) basis or on a volume per volume basis (v/v), unless stated otherwise.

In another embodiment, an oral liquid loratadine composition includes any suitable controlled-release liquid formulation available to those of ordinary skill in the art. For instance, an oral controlled release liquid formulation may provide for controlled or sustained release of the active agent, such as loratadine, from a gel, matrix, capsule, or resin material, or any combination of controlled or sustained release technology available to those of ordinary skill in the art that can be suspended or dissolved in a liquid formulation. Examples of conventional controlled or sustained-release technology that can be adapted for use with the loratadine formulations of the invention include, but are not limited to, the following. The preparation of an oral controlled release liquid formulation that releases the active agent from a semi-solid gel-like matrix has been described in U.S. Pat. No. 4,717,713, the contents of which is incorporated herein by express reference thereto. In another example, a drug-resin complex suitable for incorporation into a liquid sustained release formulation has been described in U.S. Pat. No. 4,788,055, the contents of which is incorporated herein by express reference thereto.

The present invention also relates to methods of preparing oral liquid loratadine compositions. Typically, this includes dispersing or dissolving an amount of loratadine, or a pharmaceutically acceptable salt or metabolite thereof, preferably in an effective amount, into a portion of a liquid pharmaceutically acceptable carrier to form a liquid loratadine composition, such as an oral liquid loratadine solution. The portion may be the entire carrier, a subdivided portion of the carrier, or only one or more of the carrier components, e.g., the chelating agent and the mono- or poly-hydroxy phenol component. The remainder of the carrier, if any, would then be provided subsequently by addition to the loratadine and first portion of the carrier or by separate combination and then addition of the remaining carrier as a whole to the loratadine and first portion of the carrier. The liquid loratadine composition is also combined with additional pharmaceutically acceptable carrier sufficient to form the oral liquid loratadine solution. Alternatively, all the carrier components can be combined to form a liquid into which the loratadine is dissolved to form the inventive solutions.

Preferably, a stabilizing agent, flavoring agent, sweetening agent, preservative agent, solubilizing agent, or combination thereof, are included in the carrier. In one embodiment, a substantially stable oral liquid loratadine composition may be prepared using one or more aqueous solvents. Particularly where the oral liquid loratadine composition is a solution, the solution is at least translucent and more preferably is clear or essentially clear, i.e., transparent. By “clear” is meant that the light transmission through the composition is typically at least about 70 percent, preferably at least about 90 percent, and more preferably at least about 95 percent. In an exemplary embodiment, the composition is substantially transparent to the naked eye so that substantially all the light transmits therethrough. In another embodiment, a substantially stable oral liquid loratadine composition may be prepared as a syrup, which can be administered “as is” or reconstituted to a solution or more diluted syrup or other liquid formulation.

In another embodiment, a substantially stable liquid loratadine solution may include degradation of loratadine in the composition over a period of up to about three months at about 40° C. that is no more than about 4 percent (wherein the degradation of loratadine is measured on a weight/volume basis, and further wherein the percent degradation is determined on an area/area basis), preferably no more than about 3 percent, and more preferably no more than about 2 percent, of the amount of loratadine originally present in the composition. The term “area/area basis” refers to the total area of the observed degradation peaks on a chromatogram, e.g., HPLC, divided by the total area of the loratadine peak (and multiplied by 100). In each of these embodiments, it is more preferred that the above-noted loratadine degradation is over a period of at least three months. In one embodiment, degradation of loratadine over a period of up to two months at about 40° C. is no more than about 1 percent to 2 percent (w/v) of the amount of loratadine originally present. In another embodiment, degradation of loratadine over a period of up to three months at about 40° C. is no more than about 1 percent to 2 percent (w/v) of the amount of loratadine originally present. In yet another embodiment, degradation of loratadine over a period of one month at about 40° C. is no more than about 0.5 percent (w/v) of the amount of loratadine originally present.

The present invention also surprisingly provides packaging for an oral liquid loratadine solution that is substantially stable, or preferably entirely stable, by increasing the total fill volume of the solution in a container to further reduce oxidative degradation of the loratadine due to oxygen present in the headspace remaining after conventional packaging in a typical liquid formulation bottle. In another embodiment, an oral liquid loratadine solution is provided in a substantially non-permeable container, such as a glass container, to reduce the exchange of air with oxygen and thus reduce oxidative degradation of the loratadine. Preferably, the container, such as a glass container, is not clear and transparent, but is only translucent (e.g., amber colored) or is opaque.

It is believed that reducing the unfilled volume or headspace in a container may reduce, and preferably substantially reduce, the exposure of a loratadine solution to one or more sources of oxidative degradation, for example, oxygen or oxygen-derived free radicals. Moreover, packaging a loratadine solution in a substantially non-permeable container, for example, a glass or amber glass container, may reduce and preferably substantially reduce the exchange that may occur of oxygen with air that is present within the headspace of the container or that leeches in from outside typical air permeable containers. In such a manner, oxidative degradation of loratadine may be further reduced. Other suitable changes to product packaging, types of containers, or other conditions in which the loratadine compositions are packaged or stored that are available to those of ordinary skill in the art may also be employed to further minimize, or substantially eliminate, the oxidative degradation of loratadine.

The phrase “therapeutically” in connection with the effective amount includes that amount of loratadine, alone or in combination with another active ingredient, that provides a therapeutic benefit in the treatment or management of symptoms or conditions associated with the effects of histamine, including the relief of nasal and non-nasal symptoms of seasonal allergic rhinitis, or for the treatment of chronic idiopathic urticaria, or one or more other conditions or symptoms associated therewith. The term “prophylactically” in connection with the effective amount includes that amount of loratadine that, alone or with another active ingredient, inhibits or prevents histamine release or symptoms associated therewith.

The present invention also provides methods of preventing, treating, or managing symptoms or conditions associated with the effects of histamine, such as seasonal allergic rhinitis, or conditions associated with chronic idiopathic urticaria, for example, preferably in a mammal. As used herein, the terms “preventing, treating, or managing” cover preventing, treating, or managing the specified disease in a mammal, more preferably a human, and includes: (i) preventing the disease from occurring in a subject that may be predisposed to the disease but has not yet been diagnosed as having it; (ii) inhibiting the disease, i.e., arresting its development before or after it afflicts a patient; or (iii) relieving the disease, i.e., causing regression of the disease. As used herein, “mammal” is meant the class of warm-blooded vertebrate animals that have, in the female, milk-secreting organs for feeding the young. Mammals include humans, apes, many four-legged animals, whales, dolphins, and bats. It should also be understood that symptoms of any disease are also encompassed within the term “managed,” such that managing symptoms of seasonal allergic rhinitis, for example, may address some or all of the symptoms thereof with or without actually affecting the underlying disease itself.

The methods of the invention include administering to a patient, preferably a mammal, an effective amount of an oral liquid loratadine solution of the invention. These methods find utility in preventing, treating, or managing numerous disease states and conditions that are currently being addressed with, e.g., solid dosage forms of loratadine but with an expected increase in patient compliance, and include, for example, treating patients with seasonal allergic rhinitis, and treating patients having conditions associated therewith or preventing or treating allergic symptoms in different patient populations, such as children and geriatric populations, that cannot or tend not to take or have difficulty taking solid dosage forms.

The effective amount of loratadine will vary depending on the subject being treated, the severity of the disease state and the manner of administration, and may be determined routinely by one of ordinary skill in the art. The dose, and perhaps dose frequency, will also vary according to the age, body weight, and response of the individual. The total daily dose range may preferably include, for example, from about 0.1 mg/5 mL to 20 mg/5 mL of loratadine in the oral liquid solution. Preferably, the solution will be administered in single or divided doses orally, and preferably the total daily dose of loratadine is from about 0.1 mg/5 mL to 20 mg/5 mL of the oral liquid solution. It may be necessary to use dosages outside the above ranges in some cases, as will be apparent to those of ordinary skill in the art. Further, it is noted that the clinician or treating physician will know the appropriate daily dose, and how and when to interrupt, adjust, or terminate therapy in conjunction with individual patient response.

The methods of the present invention also contemplate the addition of one or more therapeutic agents with the loratadine to provide an additive, more complete, or synergistic effect in preventing, treating, or managing a condition or disease as noted herein, or any other disease or condition for which the same patient may require prevention, treatment, or management thereof. For instance, one or more therapeutic agents may be administered to prevent, treat, or manage one or more conditions associated with an allergic response. In one embodiment, a method of preventing, treating, or managing an allergic condition or disease in a mammal includes administering to the patient a pharmaceutically effective amount of an oral liquid loratadine composition of the invention. The additional “therapeutic agents,” which may be prophylactic, therapeutic, or help manage, e.g., an allergic or allergic-related condition or disease, may be administered in any dosage form(s) suitable for the formulation as are well known in the art. Such dosage forms include, for example, solid dosage forms, such as tablets, capsules, powders, and cachets, or liquid dosage forms, such as suspensions, syrups, solutions, and elixirs. The agent may be incorporated in the loratadine liquid solution or may be administered in a separate dosage form, but are preferably another liquid dosage form. The dosage form containing the additional agent to be administered will, in any event, contain a quantity of the additional therapeutic agent(s) in an amount effective to alleviate or manage the symptoms or condition of the subject being treated or to provide a prophylactic effect. The selection of these additional therapeutic agents will depend upon the specific disease state being treated, some of which are described in detail below. Preferably, all active ingredients will be in an oral liquid form, e.g., an oral solution or suspension, more preferably in a combined form to facilitate patient compliance. When not in combined form, they can be administered concurrently or sequentially.

The oral liquid compositions of the present invention can be administered in connection with combination therapy regimens, e.g., for preventing, treating, or managing the symptoms of seasonal allergic rhinitis or hay fever, which may include nasal congestion and sneezing. For those embodiments of the invention where the loratadine liquid composition is administered with another agent effective for treating the symptoms of seasonal allergic rhinitis or hay fever, for example, and depending on the needs of the individual patient as determined by a clinician or treating physician, such additional therapeutic agents may include, for instance, one or more over-the-counter effective agents such as pseudoephedrine (e.g., Sudafed®, Pfizer), or phenylephrine hydrochloride (e.g., Neosynephrine®, Abbott) or any combination thereof.

The oral liquid loratadine compositions of the present invention can also be administered in connection with an analgesically effective amount of ibuprofen, a decongestant-effective amount of pseudoephedrine, or a combination thereof, in a pharmaceutically acceptable carrier. Such compositions may provide for the symptomatic relief of cough, cold, cold-like and flu-related symptoms and conditions by the administration of appropriate dosages of the pharmaceutical compositions. Cold and cold-like symptoms as used herein may include, for example, coryza, nasal congestion, upper respiratory infections, allergic rhinitis, otitis, and sinusitis. For example, ibuprofen (e.g., Ibuprofen®, McNeil) or pseudoephedrine (Sudafed®; Pfizer) can be administered. Other analgesic or decongestant compounds, or a combination thereof, may also or alternatively be included in the present loratadine liquid compositions.

The loratadine liquid compositions may also include one or more other classes of pharmaceutical active agents for the prevention, treatment, or management of other conditions, as deemed necessary or desired by a physician. Such other conditions may occur, for example, in patients that are also suffering from one or more cough, cold, cold-like and flu-related symptoms. Other conditions may include, but are not limited to, for example, allergic rhinitis and asthma. Loratadine may be used as adjunctive treatment with one or more anti-inflammatory medications, such as montelukast (e.g., Singulair®, Merck), for the treatment of symptoms associated with asthma and allergic rhinitis. Loratadine may also be used as adjunctive treatment with one or more corticosteroids, such as betamethasone (e.g., Diprolene®, Schering) for the treatment of allergic rhinitis or for symptoms associated with asthma.

The term “pharmaceutically acceptable salt(s)” or “a pharmaceutically acceptable salt thereof” refers to salt(s) prepared from pharmaceutically acceptable non-toxic acid or bases including inorganic acids and bases and organic acids or bases. The pharmaceutically acceptable salts used in the present invention may be amphoteric, may be present in the form of internal salts, or both.

The term “about,” as used herein, should generally be understood to refer to both numbers in a range of numerals. Moreover, all numerical ranges herein should be understood to include each whole integer within the range.

The term “substantially” means, e.g., not entirely complete, or not entirely absolute. Typically, “substantially” should be understood to refer to at least about 90 percent, preferably at least about 95 percent, and more preferably at least about 99 percent. In one more preferred embodiment, “substantially” can refer to at least about 99.5 percent or 99.9 percent. In one example, a composition that is “substantially stable,” such as an oral liquid formulation of loratadine having substantial stability, encompasses a solution that may not necessarily exhibit absolute or 100% stability over a defined period of time; instead, the composition may exhibit nearly total stability, such as greater than about 97% stability or 99.8% stability, over a particular period of time under ambient conditions (unless specified otherwise).

Conversely, “substantially free” means, e.g., almost entirely devoid of the referenced characteristic. Typically, “substantially free” should be understood to refer to less than about 5 percent, preferably less than about 1 percent, and more preferably less than about 0.1 percent. In a more preferred embodiment, it refers to less than about 0.05 percent, or less than about 0.01 percent. In one most preferred embodiment, the term refers to less than an analytically detectable amount.

The term “amount” includes both a dry quantity of an agent, compound, or component, such as a quantity that is measured or given in milligram (mg) units, as well as a quantity of an agent, compound, or component that is dissolved or otherwise present in a particular volume of a solvent or other liquid reagent and expressed in terms of a concentration, such as mg/mL. The term “effective amount” includes an amount of an active pharmaceutical agent that is required to obtain prophylactic or therapeutic efficacy against a disease or condition, or a symptom thereof, or to manage a disease or condition, or a symptom thereof. For instance, an “effective amount” or “pharmaceutically effective amount” of loratadine includes an amount of loratadine, or a salt or metabolite thereof, that is required to obtain efficacy to prevent, treat, or manage an allergic condition, or the formation or retention thereof, or the symptoms or conditions associated with an allergic condition. The term “manage” includes any action that results, for instance, in the amelioration of allergic symptoms, or a condition associated therewith, or other therapeutic effect that improves the health or well-being of a patient such as the prevention or reduction of histamine release.

Each of the patent applications, patents, publications, and other published documents mentioned or referred to in the Detailed Description is incorporated herein in its entirety by express reference thereto, to the same extent as if each was specifically and individually indicated to be incorporated by reference.

### EXAMPLES

The invention is further defined by reference to the following illustrative (non-limiting) examples, describing in detail specific excipients, in addition to storage and solubility conditions, that may be used to prepare or administer the liquid formulations of the present invention.

### Example 1

### Sample Loratadine Liquid Formulation that Includes BHA

The following formulation, shown below, represents one example of a loratadine-containing formulation that demonstrated substantial stability, and which represents a stable oral liquid formulation. As indicated below, two concentrations of BHA were separately tested, i.e., BHA was present at 0.95 mg in one study, and BHA was present at 0.5 mg in another study. Citric acid anhydrous was included as a chelating agent. The total volume of the solution was 5 mL.

| Ingredient | Quantity/5 mL | |
|---|---|---|
| Loratadine (micronized), USP | 5 | mg |
| Purified water, USP | 1.0 | mL |
| Sodium benzoate, NF | 5 | mg |
| Citric acid anhydrous, USP | 100 | mg |
| Butylated hydroxyanisole (BHA) | 0.95 | mg (or 0.5 mg) |
| Propylene glycol, USP | 0.435 | mL |
| Glycerin, USP | 0.325 | mL |
| Flavoring agent (Natural and Artificial | 0.03 | mL |
| Fruity) | | |
| Liquid sugar | to make 5 | mL |

The surprising and unexpected effects of BHA on stability are shown in Table 1. The 0.95 mg/5 mL concentration of BHA, as indicated above, is equivalent to the 0.19 g/L concentration of BHA shown in Table 1). Moreover, the 0.1 g/L concentration of BHA, as shown in Table 1, is equivalent to 0.5 mg/5 mL BHA indicated above. As shown in Table 1, introducing BHA in the formulation with the presence of citric acid concentration yielded advantageous stability profiles at both 40° C. and 50° C. for 2 weeks as well as at 40° C./75% RH for 12 weeks. In addition, BHA at a concentration of 0.1 g/L had a similar effect as the 0.19 g/L concentration of BHA. Increasing citric acid alone did not provide a similar benefit indicating that incorporating BHA in the formulation together with citric acid provided substantial protection against metal-mediated oxidation of loratadine.

The abbreviations “Imp-1” and “Imp-2” refer to specific USP impurities. The USP Specification for Impurities, which represent the specified limits for each impurity, are as follows: USP Imp-1 (≦0.3%); USP Imp-2 (≦0.3%); and any other individual impurities (≦0.2%). Moreover, these formulations were each tested in a PET container.

TABLE 1*

| | | | | Two weeks | | | | | | | | |
|---|---|---|---|---|---|---|---|---|---|---|---|---|
| | Initial | | | 30° C. | | | 40° C. | | | 50° C. | | |
| Approach | Imp 1 | Imp 2 | Total | Imp 1 | Imp 2 | Total | Imp 1 | Imp 2 | Total | Imp 1 | Imp 2 | Total |
| Citric acid (20 g/L) + BHA | nd | nd | 0.0245 | nd | 0.05 | 0.0739 | 0.0349 | 0.0667 | 0.1016 | 0.0534 | 0.0979 | 0.1782 |
| (0.19 g/L) | | | | | | | | | | | | |
| Citric acid (20 g/L) + BHA | nd | nd | 0.0243 | nd | 0.0568 | 0.0793 | 0.0498 | 0.0937 | 0.1435 | 0.0477 | 0.1108 | 0.1843 |
| (0.1 g/L) | | | | | | | | | | | | |
| Citric acid (20 g/L) | nd | nd | 0.0241 | 0.0395 | 0.0977 | 0.1641 | 0.1228 | 0.2268 | 0.5246 | 0.0749 | 0.126 | 0.2916 |

| | 40° C./ 75% RH | | | | | | | | | | | |
|---|---|---|---|---|---|---|---|---|---|---|---|---|
| | 2 weeks | | | 4 weeks | | | 8 weeks | | | 12 weeks | | |
| Approach | Imp 1 | Imp 2 | Total | Imp 1 | Imp 2 | Total | Imp 1 | Imp 2 | Total | Imp 1 | Imp 2 | Total |
| Citric acid | nd | 0.065 | 0.0893 | 0.0365 | 0.0888 | 0.1524 | 0.0622 | 0.1272 | 0.2177 | 0.1209 | 0.1877 | 0.5401 |
| (20 g/L) + BHA | | | | | | | | | | | | |
| (0.10 g/L) | | | | | | | | | | | | |

*The abbreviations “Imp-1” and “Imp-2” refer to specific USP impurities;
nd = not detected

### Example 2

### Stability Testing Using Different Flavors

Experiments were conducted to determine a possible correlation between a particular flavoring agent and generation of degradants of loratadine. In a preliminary set of experiments, the Natural and Fruity flavoring agent of Example 1 was replaced with one or more alternate flavors, including artificial pineapple, natural strawberry, cherry, artificial banana and natural & artificial fruit mix flavor.

Table 2 shows the stability data under stress conditions (65° C./7 days) for several flavoring agents that were tested. Based on preliminary experimental results, commercially available banana, pineapple, and strawberry flavoring agents were tested at accelerated conditions. As shown in Table 3, artificial banana flavoring agent, when used in the formulation together with a change in the container to glass showed preferred stability profiles, as compared to the other flavoring agents that were tested when stored at accelerated conditions (40° C./75% R). Data represent percentages, e.g., 0.0315 represents 0.0315%, in various tables in the Examples.

TABLE 2*^{#}

| | | | | 65° C. | | | | | |
|---|---|---|---|---|---|---|---|---|---|
| | Initial | | | 3 day | | | 7 day | | |
| Flavoring | | | Total | | | Total | | | Total |
| Agent | Imp 1 | Imp 2 | Imp | Imp 1 | Imp 2 | Imp | Imp 1 | Imp 2 | Imp |
| Natural and Fruity | nd | nd | 0.0315 | 0.0365 | 0.2722 | 0.8566 | Did not analyze | | |
| | | | | 0.0394 | 0.2637 | 0.8288 | | | |
| Artificial banana | nd | nd | 0.0293 | | 0.0667 | 0.0908 | nd | 0.0623 | 0.0865 |
| | | | | | 0.051 | 0.0787 | nd | 0.0592 | 0.0843 |
| Cherry | nd | nd | 0.0216 | 0.0544 | 0.0465 | 0.1279 | Did not analyze | | |
| | | | | 0.05 | 0.0541 | 0.1365 | | | |
| Natural and | nd | nd | 0.0238 | 0.0219 | 0.1507 | 0.4522 | | | |
| Artificial mix fruit | | | | 0.0235 | 0.1609 | 0.4772 | | | |
| Pineapple | nd | nd | 0.0354 | | 0.0765 | 0.1136 | nd | nd | 0.0297 |
| | | | | | 0.0676 | 0.1042 | nd | 0.0774 | 0.1095 |
| Strawberry | nd | nd | 0.0238 | | 0.0417 | 0.0667 | 0.027 | 0.0554 | 0.1083 |
| | | | | | 0.0513 | 0.0804 | 0.0262 | 0.0557 | 0.1057 |

^{#}Limit of Quantification (L.O.Q) ≦0.1%. All impurities below L.O.Q are also reported.
*The abbreviations “Imp-1” and “Imp-2” refer to specific USP impurities;
nd = not detected

TABLE 3*

| | 40° C./75% RH | | | | | | | | | | | |
|---|---|---|---|---|---|---|---|---|---|---|---|---|
| | 2 weeks | | | 4 weeks | | | 8 weeks | | | 12 weeks | | |
| Flavoring | | | Total | | | Total | | | Total | | | Total |
| Agent | Imp 1 | Imp 2 | Imp | Imp 1 | Imp 2 | Imp | Imp 1 | Imp 2 | Imp | Imp 1 | Imp 2 | Imp |
| Artificial | ND | 0.0291 | 0.0828 | 0.0155 | 0.0275 | 0.0974 | ND | 0.0357 | 0.0610 | 0.0296 | 0.0461 | 0.1367 |
| banana | | | | | | | | | | | | |
| Pineapple | 0.0137 | 0.0291 | 0.0922 | 0.0193 | 0.0402 | 0.1125 | 0.0175 | 0.0314 | 0.0754 | 0.0367 | 0.0662 | 0.1590 |

| | | | | |
|---|---|---|---|---|
| Strawberry | 0.0173 | 0.0375 | 0.1331 | Study discontinued |

*The abbreviations “Imp-1” and “Imp-2” refer to specific USP impurities;
nd = not detected

### Example 3

### Effect of Packaging Parameters

Both the headspace and the packaging components were determined to surprisingly affect degradant generation.

The extent of degradant generation as a function of headspace was studied under stress conditions at 65° C./3 days for a loratadine oral solution. The percent of Imp-1, Imp-2 and total impurities as a function of headspace is shown in Table 4. An increase in headspace resulted in a concomitant increase in Imp-1, Imp-2 and total impurity levels.

TABLE 4*^{#}

| | Impurity (%) | | |
|---|---|---|---|
| Headspace (%) | IMP-1 | IMP-2 | TOTAL |
| 0 | 0.0380 | 0.0720 | 0.1389 |
| 11 | 0.0691 | 0.0956 | 0.2544 |
| 25 | 0.0831 | 0.1050 | 0.2951 |
| 50 | 0.1120 | 0.1299 | 0.3788 |
| 75 | 0.1891 | 0.2057 | 0.7074 |

^{#}Limit of Quantification (L.O.Q) ≦0.1%. All impurities below L.O.Q are also reported.
*The abbreviations “Imp-1” and “Imp-2” refer to specific USP impurities.

Based on these observations, it was determined that using a fill volume of liquid formulations of the invention can be increased in the final product packaging to an excess fill volume to reduce the headspace in the container. Glass bottles with such excess fill volume, i.e., with reduced head space, also showed significantly better impurity profiles at 40° C. over 12 weeks compared to PET counterparts at 40° C. after 12 weeks.

### Example 4

### Another Sample Loratadine Liquid Formulation that Includes BHA

As compared to the formulation shown in Example 1, the loratadine-containing formulation (e.g., solution) shown below included BHA at a reduced level, as well as a reduced level of citric acid, together with an alternate flavoring agent, artificial banana. The total volume of the solution was 5 mL.

| Ingredient | Quantity/5 mL | |
|---|---|---|
| Loratadine (micronized), USP | 5 | mg |
| Purified water, USP | 1.0 | mL |
| Sodium benzoate, NF | 5 | mg |
| Citric acid anhydrous, USP | 65 | mg |
| Butylated hydroxyanisole (BHA) | 0.375 | mg |
| Propylene glycol, USP | 0.435 | mL |
| Glycerin, USP | 0.325 | mL |
| Flavoring agent (Natural and Artificial Fruity) | 0.03 | mL |
| Liquid sugar | to make 5 | mL |

This formulation can provide surprising and unexpected stability of loratadine according to the invention compared to conventional liquid formulations that, e.g., do not include BHA, use different flavoring agents that more rapidly degrade the stability of loratadine, etc.

### Example 5

### Accelerated Stability Data for Formulation with BHA of Invention

A loratadine oral solution formulation was filled into 4 ounce amber glass bottles with excess fill volume and placed under accelerated stability conditions at 40° C. and 75% RH. The accelerated stability results for the formulation are shown in Table 5.

As shown in Table 5, at time zero and at 4 weeks, the USP impurities Imp-1 and Imp-2 were not detected, while the total impurities were 0.056% and 0.032%, respectively. The USP impurities Imp-1 and Imp-2 were observed after 8 weeks at 0.02% and 0.0397%, respectively, with total impurities at 0.0998%. The USP impurities along with total impurities at the 12 week time station, e.g., after 12 weeks, were 0.0154%, 0.0345% and 0.1071%, respectively. The degradant values after 12 weeks were well within the USP specifications of 0.3% for Imp-1 and Imp-2 and 0.5% for total impurities.

TABLE 5*

| | | Initial | 40° C./75% RH | | | | | | | | |
|---|---|---|---|---|---|---|---|---|---|---|---|
| | Container | Total Imp | 4 weeks | | | 8 weeks | | | 12 weeks | | |
| Approach | Fill | (Imp 1&2 ND) | Imp 1 | Imp 2 | Total Imp | Imp 1 | Imp 2 | Total Imp | Imp 1 | Imp 2 | Total Imp |
| Control batch | GLS | 0.0304 | 0.0285 | 0.0258 | 0.1917 | 0.0425 | 0.0251 | 0.1785 | 0.1035 | 0.1024 | 0.4008 |
| (no change) | Increased | | | | | | | | | | |
| | fill | | | | | | | | | | |
| BHA | GLS | 0.0555 | ND | ND | 0.0323 | 0.02 | 0.0397 | 0.0998 | 0.0154 | 0.0345 | 0.1071 |
| and 0.6% | Increased | | | | | | | | | | |
| banana flavor | fill | | | | | | | | | | |

*The abbreviations “Imp-1” and “Imp-2” refer to specific USP impurities.
The term “GLS Increased fill” refers to “Glass Increased fill.”

### Example 6

### Stability Evaluation Under Accelerated and Controlled Room Temperature Conditions

A batch formulation including BHA and an alternate flavor was prepared. BHA was included in the formulation at a concentration of 0.075 g/L. The amount of citric acid used in the formulation was increased from 8 g/L to 13 g/L compared to prior tests to facilitate the solubility of BHA in the solution. Artificial banana was used as the flavoring agent at a concentration of 6 mL/L. The stability data, as shown below in Table 6, demonstrated that the addition of BHA to the formulation significantly lowered the degradant profiles compared to other approaches that were tested.

During testing, the formulation was also packaged in two different container-closure configurations. The details of each container-closure configuration is as follows:

BTL, 4 ounce PET 24/400 AMBER B/R with CAP Wht PP CRC W/PE liner, CRC 24/400

BTL, 4 ounce GLS 22/405 AMBER B/R with CAP 22/400 Wht PP CRC W/PE LNR

Each of the configurations was placed at accelerated and control room temperature conditions as shown below. The samples obtained were tested at time intervals in accordance with FDA Guidelines.

Accelerated Stability Conditions

Storage conditions: 40°±2° C.

Orientation: Horizontal and Upright

Samples tested at the following intervals: 4, 8, and 12 weeks

Controlled Room Temperature Conditions

Storage conditions: 25°±2° C.

Orientation: Horizontal and Upright

Samples tested at the following Intervals: 0, 3, 6, 9, 12, 18, and 24 months

The stability results are shown in Table 6. Table 6 shows the stability results under accelerated conditions, at 40° C. and 75% RH. The formulations included BHA and artificial banana (as the flavoring agent), and was stored in containers with increased fill volume.

As shown in Table 6, at time zero, the USP impurities were not detected while the total impurities were below the limit of quantification. The degradant levels at the 12-week time point were 0.0183%, 0.0409% and 0.0592% for Imp-1, Imp-2 and total impurities, respectively, for samples packaged in 4 ounce amber glass containers with reduced headspace and oriented upright in the stability chamber. Samples oriented in a horizontal orientation showed similar degradant values. For example, horizontally placed samples after 12 weeks showed 0.0175%, 0.0430% and 0.0605% for Imp-1, Imp-2 and total impurities, respectively. All the unknown degradants for both the horizontal and upright samples were below the limit of quantification and were not reported. All the degradant values for both the upright and the horizontal samples after 12 weeks were within the specified limits as set by the USP. The values shown in Table 6 were determined at different relative retention times (or “RRT”).

TABLE 6^{#}
Time = 0

| RRT | USP IMP-1* | USP IMP-2* | RRT 0.50 | RRT 0.66 | RRT 0.96 | RRT 1.58 | RRT 1.69 | TOTAL** |
|---|---|---|---|---|---|---|---|---|
| Impurity | ND | ND | Below LOQ | Below LOQ | Below LOQ | Below LOQ | Below LOQ | 0.000 |

T = 4 weeks

| RRT | USP IMP-1 | USP IMP-2 | RRT 0.66 | RRT 1.32 | RRT 1.57 | TOTAL** |
|---|---|---|---|---|---|---|
| Upright | ND | 0.0125 | Below LOQ | Below LOQ | Below LOQ | 0.0125 |
| Horizontal | ND | 0.0225 | Below LOQ | Below LOQ | Below LOQ | 0.0225 |

T = 8 weeks

| RRT | USP IMP-1* | USP IMP-2* | RRT 0.50 | RRT 0.65 | RRT 0.93 | RRT 1.30 | RRT 1.45 | RRT 1.54 | RRT 1.60 | TOTAL** |
|---|---|---|---|---|---|---|---|---|---|---|
| Upright | 0.0173 | 0.0338 | Below | Below LOQ | Below LOQ | Below LOQ | Below LOQ | Below LOQ | Below LOQ | 0.0511 |
| | | | LOQ | | | | | | | |
| Horizontal | 0.0202 | 0.0405 | Below | Below LOQ | Below LOQ | Below LOQ | Below LOQ | Below LOQ | Below LOQ | 0.0607 |
| | | | LOQ | | | | | | | |

T = 12 weeks

| RRT | USP IMP-1* | USP IMP-2* | RRT 0.49 | RRT 0.65 | RRT 0.96 | RRT 1.39 | RRT 1.49 | RRT 1.56 | RRT 1.66 | TOTAL** |
|---|---|---|---|---|---|---|---|---|---|---|
| Upright | 0.0183 | 0.0409 | Below | Below LOQ | Below LOQ | Below LOQ | Below LOQ | Below LOQ | Below LOQ | 0.0592 |
| | | | LOQ | | | | | | | |
| Horizontal | 0.0175 | 0.043 | Below | Below LOQ | Below LOQ | Below LOQ | Below LOQ | Below LOQ | Below LOQ | 0.0605 |
| | | | LOQ | | | | | | | |

USP Specifications for Impurities:
*USP Imp-1 (≦0.3%); USP Imp-2 (≦0.3%); and any other individual impurities (≦0.2%).
^{#}The abbreviations “Imp-1” and “Imp-2” refer to specific USP impurities;
**Total impurities ≦0.5%
Limit of Quantification (L.O.Q.) ≦0.1%; All impurities except USP Imp-1 and USP Imp-2 below L.O.Q. were not reported.
The abbreviation “RRT” refers to Relative Retention Time.

Although each of the formulations described above includes specific amounts or concentrations of different excipients, the amount or concentration of one or more of the excipients may be varied as needed or desired so long as the stability of the oral liquid loratadine formulation is not significantly adversely impacted.

Further, although preferred embodiments of the invention have been described in the foregoing description, it will be understood that the invention is not limited to the specific embodiments disclosed herein but is capable of numerous modifications by one of ordinary skill in the art. It will be understood that the materials used and the chemical or pharmaceutical details may be slightly different or modified from the descriptions herein without departing from the methods and compositions disclosed and taught by the present invention.

What is claimed is:

1. An oral liquid formulation comprising:
a therapeutically or prophylactically effective amount of loratadine, or a pharmaceutically acceptable salt or metabolite thereof; and
a pharmaceutically acceptable carrier comprising:
a mono- or poly-hydroxy phenol component in an amount sufficient to increase stability of the loratadine;
a solubilizing agent present in an amount sufficient to facilitate dissolution of the loratadine and the phenol component; and
a chelating agent comprising at least one organic acid in an amount sufficient to increase dissolution of the phenol component and loratadine and to increase stability of the loratadine.

2. The formulation of claim 1, wherein the phenol component comprises butylated hydroxyanisole.

3. The formulation of claim 1, wherein the sufficient amount of phenol component is from about 0.05 mg/5 mL to 5 mg/5 mL of the formulation.

4. The formulation of claim 1, wherein the organic acid comprises one or more of acetic acid, propionic acid, butyric acid, a fatty acid of 6-22 carbon atoms, bile acid, lactic acid, citric acid, pyruvic acid, oxalic acid, malic acid, malonic acid, succinic acid, maleic acid, fumaric acid, tartaric acid, benzoic acid, cinnamic acid, mandelic acid and salicylic acid.

5. The formulation of claim 1, wherein the sufficient amount of the chelating agent is from about 1 mg/5 mL to 150 mg/5 mL of the formulation.

6. The formulation of claim 1, wherein the solubilizing agent comprises a glycol present in an amount of about 4 volume percent to 15 volume percent (v/v) and the pharmaceutically acceptable carrier further comprises one or more of a stabilizing agent, thickening agent, sweetening agent, flavoring agent, colorant agent, preservative agent, a second different antioxidant agent, or buffering agent.

7. The formulation of claim 6, wherein the carrier further comprises at least one of a sweetening agent, a flavoring agent, and a preservative agent.

8. The formulation of claim 7, wherein the sweetening agent comprises glycerin, sucrose, liquid sugar, sorbitol, saccharin, xylitol, maltitol, an acesulfame-containing, sucralose-containing or saccharin-containing component, or a combination thereof; and the flavoring agent comprises grapefruit, orange, lemon, lime, mango, strawberry, banana, pineapple, cherry, or a combination thereof.

9. The formulation of claim 8, wherein the sweetening agent is present in an amount of about 1 volume percent to 85 volume percent (v/v).

10. The formulation of claim 9, wherein the sweetening agent comprises liquid sugar and glyercin.

11. The formulation of claim 7, wherein the preservative agent comprises one or more of sodium benzoate, chlorobutanol, benzyl alcohol, and benzalkonium chloride.

12. The formulation of claim 11, wherein the preservative agent is present in an amount of about 0.05 mg/5 mL to 10 mg/5 mL.

13. The formulation of claim 7, wherein the flavoring agent is present in an amount of about 0.01 volume percent to 1 volume percent (v/v).

14. The formulation of claim 7, wherein at least two of the sweetening agent, a flavoring agent, and a preservative agent are present.

15. The formulation of claim 14, wherein all of the sweetening, flavoring, and preservative agents are present and the sweetening agent comprises glycerin and liquid sugar in an amount of about 20 volume percent to 70 volume percent (v/v); the flavoring agent comprises strawberry, banana, pineapple, or a combination thereof, in an amount of about 0.01 volume percent to 1 volume percent (v/v); and the preservative agent comprises sodium benzoate in an amount of about 0.1 mg/5 mL to 10 mg/5 mL.

16. The formulation of claim 1, wherein the therapeutically or prophylactically effective amount of loratadine, or salt or metabolite thereof, is a concentration of about 0.1 mg/5 mL to 20 mg/5 mL of the formulation.

17. The formulation of claim 1, wherein the formulation is at least substantially stable.

18. The formulation of claim 1, wherein degradation of loratadine over a period of up to three months is no more than about 1 percent to 2 percent (w/v) at 40° C.

19. The formulation of claim 6, wherein the glycol comprises propylene glycol.

20. A stable oral liquid formulation comprising:
a therapeutically or prophylactically effective amount of loratadine, or a pharmaceutically acceptable salt or metabolite thereof; and
a pharmaceutically acceptable carrier comprising:
butylated hydroxyanisole;
a solubilizing agent present in an amount sufficient to facilitate dissolution of the loratadine and the butylated hydroxyanisole; and
a chelating agent comprising at least one of citric acid anhydrous, acetic acid, propionic acid, butyric acid, a fatty acid of 6-22 carbon atoms, bile acid, lactic acid, pyruvic acid, oxalic acid, malic acid, malonic acid, succinic acid, maleic acid, fumaric acid, tartaric acid, benzoic acid, cinnamic acid, mandelic acid and salicylic acid,
wherein the butylated hydroxyanisole and the chelating agent are each present in amount sufficient to synergistically increase the stability of the loratadine.

21. The stable oral liquid loratadine formulation of claim 20, wherein the effective amount of loratadine is from about 1 mg/5 mL to 20 mg/5 mL of loratadine, or a pharmaceutically acceptable salt or metabolite thereof; wherein the chelating agent includes citric acid; and further comprising a preservative agent, a sweetening agent, and a flavoring agent.

22. A method of preparing a stable oral liquid loratadine formulation which comprises:
providing a pharmaceutically acceptable carrier comprising:
a mono- or poly-hydroxy phenol component in an amount sufficient to increase stability of the formulation;
a solubilizing agent present in an amount sufficient to facilitate dissolution of the loratadine and the phenol component; and
a chelating agent comprising at least one organic acid in an amount sufficient to increase dissolution of the phenol component and to increase stability of the formulation; and
dissolving a therapeutically or prophylactically effective amount of loratadine, or a pharmaceutically acceptable salt or metabolite thereof, into a portion of the pharmaceutically acceptable carrier so as to provide the stable oral liquid loratadine formulation.

23. The method of claim 22, wherein the phenol component comprises butylated hydroxyanisole.

24. The method of claim 22, wherein the carrier comprises butylated hydroxyanisole and citric acid anhydrous.

25. The method of claim 22, wherein the oral liquid loratadine formulation is clear or translucent.

26. A method of preventing, treating, or managing allergic symptoms in a mammal which comprises administering to the mammal a therapeutically or prophylactically effective amount an oral liquid loratadine formulation prepared according to claim 22.

27. The method of claim 26, wherein the formulation is administered once or twice a day as a syrup.

28. The method of claim 26, wherein the total daily dose of loratadine is from about 5 mg to 50 mg.

29. The method of claim 26, which further comprises administering an effective amount of at least one other therapeutic agent.

30. A liquid formulation container comprising the oral liquid loratadine formulation of claim 1 disposed in a substantially non-air permeable bottle, wherein the formulation fills greater than about 90% of the bottle so as to reduce container headspace and to decrease oxidative degradation of the loratadine.
